(19) United States
(12) Patent Application Publication
(10) Pub. No.: US 2025/0172534 A1
(43) Pub. Date: May 29, 2025

(54) METHOD OF CHARACTERIZING WATER-OIL MIXTURES FROM ELECTRICAL CONDUCTIVITY MEASUREMENTS

(71) Applicants: Schlumberger Technology Corporation, Sugar Land, TX (US); Saudi Arabian Oil Company, Dhahran (SA)

(72) Inventors: Muhanned Alsaif, Dhahran (SA); Wael Abdallah, Sugar Land, TX (US); Shouxiang Ma, Dhahran (SA); Mustafa A. Huwaider, Dhahran (SA)

(21) Appl. No.: 18/521,554

(22) Filed: Nov. 28, 2023

(51) Int. Cl. G01N 33/18; G16C 20/70

(52) U.S. Cl. CPC G01N 33/1833; G16C 20/70

(57) ABSTRACT

Embodiments presented provide for a characterization of water-oil mixtures. In embodiments, electrical conductivity measurements are used to characterize water-oil mixtures.

### CROSS-REFERENCE TO RELATED APPLICATIONS

None.

### FIELD OF THE DISCLOSURE

Aspects of the disclosure relate to characterizing water-oil mixtures commonly found during hydrocarbon recovery operations. More specifically, aspects of the disclosure relate to water-oil mixtures based upon electrical conductivity measurements.

### BACKGROUND

Formation water salinity is an important parameter in many aspects of reservoir characterization. For reservoir saturation assessment, formation water salinity is needed as an input parameter when interpreting resistivity logs. While resistivity tools are robust and offer representative deep reservoir measurements, the interpretations of the data from the resistivity tools are sensitive to formation water salinity, i.e., the amount of total dissolved salts. Resistivity logs must therefore be paired with formation water salinity values for proper log interpretations. Without such pairing, the overall data obtained may be compromised.

To characterize formation water salinity, the most common approach is to collect produced water samples at the surface and analyse the water sample properties in a water geochemistry laboratory. As will be understood, such an analysis is only a first order of approximation of the values needed.

A second approach may be used to characterize the needed values. In this approach, water samples are collected from the downhole environment using a downhole fluid sampler. The collected sample may then be analysed for the properties needed. Such downhole water samples; however, may still not be representative of formation water.

The third approach is to directly take water samples from the formation by using a formation testing and sampling tool. Although the operation is typically expensive, and sampling clean formation water without drilling fluid contamination can be very challenging, the results can be encouraging. Currently, researchers are investigating extracting formation water salinity from chlorine logs. To be more practical and economic, it is desirable to derive formation water salinity from conventional production logs via inversion. The presence of oil-in-water in the region of interest; however, will highly alter the measurement as the oil is known to be non-conductive. To overcome this problem, the relationship between water-oil mixtures and salinity should be well characterized.

There is a need to provide an apparatus and methods that are easier to operate than conventional apparatus and methods that would allow for characterization of water-oil mixtures obtained from hydrocarbon recovery operations.

There is a further need to provide apparatus and methods that do not have the drawbacks discussed above, namely the inaccuracy of evaluations that are inherent in the analysis of downhole fluids.

There is a still further need to reduce economic costs and carbon footprint associated with operations and apparatus described above with conventional tools in the characterization of water-oil mixtures.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are; therefore, not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a method is disclosed comprising obtaining at least one electrical conductivity measurement for a downhole sample. The method may further comprise obtaining at least one temperature measurement for the downhole sample. The method may further comprise calculating an oil-in-water fraction for a water salinity value of zero. The method may further comprise using the oil-in-water fraction for a water salinity value of zero, running three models to produce a first set of data, a second set of data, and a third set of data, wherein the first set of data is related to a first model, the second set of data is related to a second model, and the third set of data is related to a third model. The method may further comprise calculating a final oil-in-water fraction using the first set of data, the second set of data, and the third set of data. The method may also comprise calculating a final water salinity using the first set of data, the second set of data, and the third set of data.

In another example embodiment, an object of manufacture configured with a non-volatile memory is disclosed wherein the non-volatile memory is configured to store a list of instructions, the list of instructions configured to be read by a computer, the list of instructions comprising, at least in part, a method. The method may comprise obtaining at least one electrical conductivity measurement for a downhole sample. The method may also comprise obtaining at least one temperature measurement for the downhole sample. The method may also comprise calculating an oil-in-water fraction for a water salinity value of zero. The method may also comprise using the oil-in-water fraction for a water salinity value of zero, running three models to produce a first set of data, a second set of data, and a third set of data, wherein the first set of data is related to a first model, the second set of data is related to a second model, and the third set of data is related to a third model. The method may also comprise calculating a final oil-in-water fraction using the first set of data, the second set of data, and the third set of data. The method may also comprise calculating a final water salinity using the first set of data, the second set of data, and the third set of data.

In another example embodiment, a method is disclosed. In this example embodiment, the method may comprise obtaining at least one electrical conductivity measurement for a downhole sample. The method may also comprise obtaining at least one temperature measurement for the downhole sample. The method may also comprise calculating an oil-in-water fraction for a water salinity value of zero. The method may also comprise using the oil-in-water fraction for a water salinity value of zero, running three models to produce a first set of data, a second set of data, and a third set of data, wherein the first set of data is related to a first model, the second set of data is related to a second model, and the third set of data is related to a third model. The method may also comprise calculating a final oil-in-water fraction using the first set of data, the second set of data, and the third set of data and a water salinity value. The method may also comprise calculating a final water salinity using the first set of data, the second set of data, and the third set of data and a oil-in-water fraction.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are; therefore, not be considered limiting of its scope, for the disclosure may admit to other equally effective embodiments.

FIG. 1 is a graph of oil-in-water data in relation to conductivity measurements for a hydrocarbon-based recovery.

FIG. 2 is a graph of normalized conductivity versus water phase salinity for various oil fractions.

FIG. 3 is a graph of interpolation and extrapolation results of electrical conductivity plotted by oil fractions.

FIG. 4 is a graph of an overall workflow of apparent salinity estimation in accordance with one example embodiment of the disclosure.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments, and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”, “second”, and other numerical terms, when used herein, do not imply a sequence or order unless clearly indicated by the context. Thus, a first element, component, region, layer or section discussed herein could be termed a second element, component, region, layer or section without departing from the teachings of the example embodiments.

When an element or layer is referred to as being “on,” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood; however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

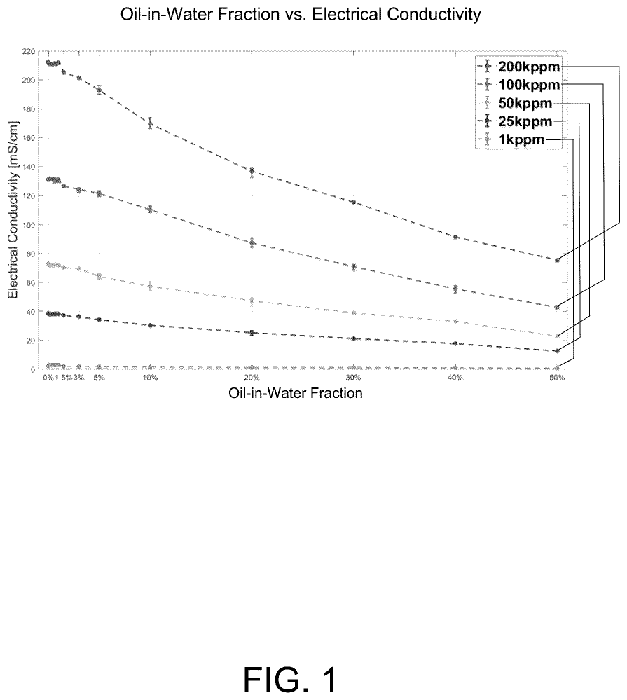

Aspects of the disclosure present a method to quantify fluid salinity from electrical conductivity measurements of water-oil mixture (WOM) knowledge. The relationships between oil-in-water (OiW) fraction with conductivity measurements are shown in FIG. 1 with fluids of multiple salinities. For all salinities, the electrical conductivity experiences its first noticeable drop with OiW at 1.5 percent, and then starts to decrease as OiW fraction increases. This change can be approximated as linear, up to a maximum of tested OiW of 50 percent, for all salinities. In embodiments, the higher the water salinity, the higher the electrical conductivity of the WOM samples at a given OiW fraction. Experimental results show no crossover between salinity lines. This establishes the possibility to estimate water phase salinity from electrical conductivity measurements even with the presence of oil content in the water, i.e., water sample contaminated by oil. As illustrated in FIG. 1, five different sets of data are presented. The sets of data include a 1 kPPM data set, a 25 kPPM data set, a 50 kPPM data set, a 100 kPPM data set, and a 200 kPPM data set. As will be understood, other data sets, such as different value data sets, may be used in embodiments of the disclosure.

From the top down in the graphs presented, the data sets illustrated are for 200 kPPM, 100 kPPM, 50 kPPM, 25 kPPM and 1 kPPM. For the 200 kPPM data set at the top, electrical conductivity values range from approximately 200 mS/mm to a low value of 80 mS/mm. For the 100 kPPM data set (second from top), electrical conductivity values range from approximately 130 mS/mm to a low value of 60 mS/mm. For the 50 kPPM data set (third from top), electrical conductivity values range from approximately 70 mS/mm to a low value of 30 mS/mm. For the 25 kPPM data set (fourth from top), electrical conductivity values range from approximately 40 mS/mm to a low value of 17 mS/mm. For the 1 kPPM data set (fifth from top), electrical conductivity values range from approximately 3 mS/mm to a low value of 1 mS/mm. As will be understood, the above values are approximate and may vary between individual cases.

Such oil dampening effect can be described quantitively by computing the linear approximation of each of the curves. Once computed, the results show that the slope monotonically decreases as OiW fraction increases, which means the conductivity and salinity dependency gets weaker as oil fraction increases. Conversely, as the oil fraction decreases, the conductivity and salinity dependency become stronger.

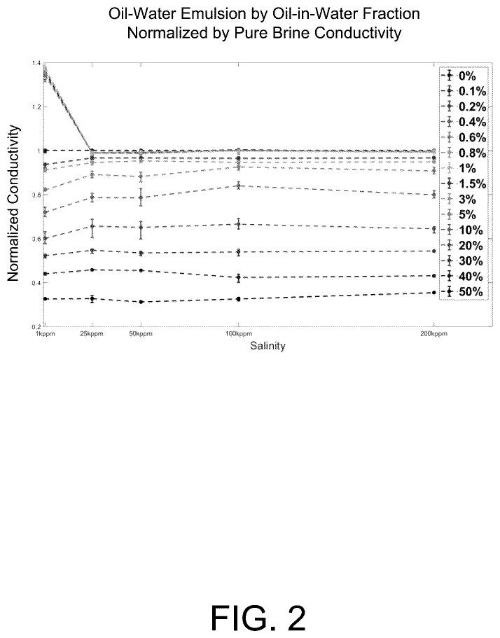

To better understand the conductivity difference in terms of OiW percentage, the relationship of FIG. 1 is replotted where conductivity measurements are normalized by the pure brine measurements (i.e., each salinity measurement is normalized by its 0 percent OiW measurement), see FIG. 2 versus water phase salinity. The first curve is flat and start from unity as each salinity is normalized by its pure brine measurements. As OiW increases above 1.5 percent, the curve is shifted downward. Also, all the curves remain approximately parallel to each other. This means OiW affects the conductivity of all salinity in a similar manner.

Referring to FIG. 1, it is of special note that water salinity of 1 kppm is considered fresh water. Treating the water salinity as fresh water is a difficult problem for subsurface formation evaluation. Electrical conductivity measurements of fresh water are small (FIG. 1) and can carry large uncertainties after normalization, as evidenced by FIG. 2.

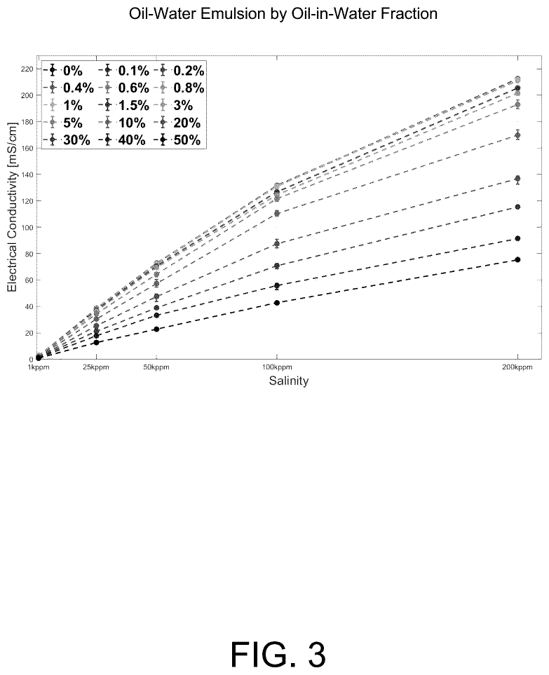

When the salinity and WOM electrical conductivity relationship is established, it is then possible to derive an analytical solution to estimate the apparent salinity given OiW and electrical conductivity measurements. In a case where insufficient measurements are available, the normalized measurements are averaged and then extrapolated to higher OiW to determine the zero-conductivity point. In the instant case, this value is approximately 71 percent. Using this zero-intersection as an additional point for each salinity, the curves are extrapolated to OiW from 50 percent to 100 percent. After this extrapolation, all the data points are jointly interpolated for intermediate salinities using cubic spline interpolation, which is a piecewise cubic polynomial that enforces continuity and smoothness at all data points. FIG. 3 shows the interpolation and extrapolation results of electrical conductivity plotted by OiW fraction.

In one non-limiting example, for water phase salinity of 100 kppm, 1.5 percent OiW will reduce conductivity by about 5 percent, 10 percent OiW reduces conductivity by about 17 percent, and 50 percent OiW reduces conductivity by about 67 percent.

Alternatively, for another example at OiW 20 percent, reduction of conductivity is about 35 percent for all water phase salinities.

As shown in FIG. 3, as the salinity of the solution increases from the origin point to the right, the electrical conductivity increases in a roughly straight-line relationship up to a salinity of 100 ppm. After reaching the 100 ppm value, the slope of the linear relationship changes up to the 200 ppm values.

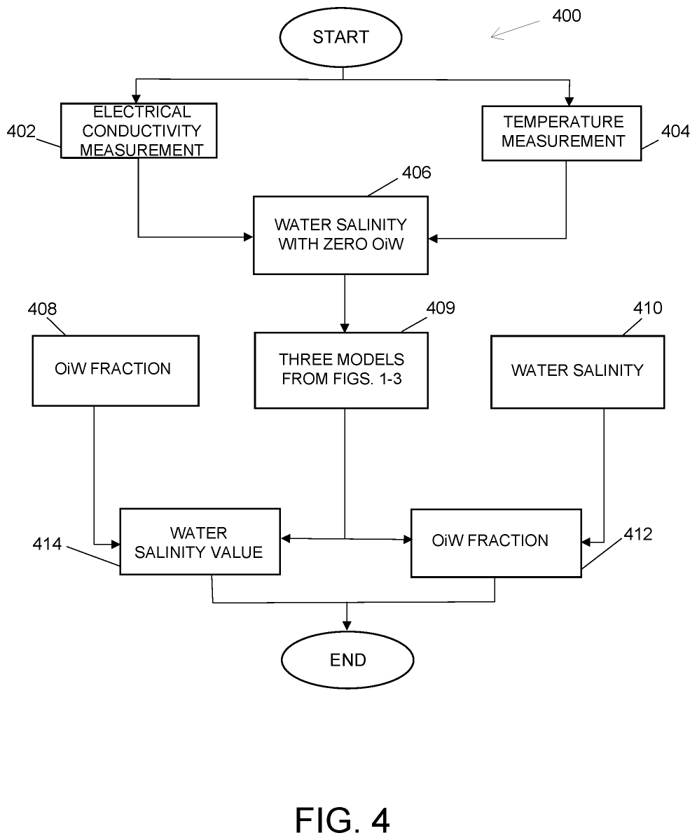

Referring to FIG. 4, a workflow for obtaining a salinity estimation is illustrated. Variations of the workflow may be permitted. The method 400 illustrated uses a electrical conductivity measurement 402 as well as a temperature measurement 404 in order to perform the method. Electrical conductivity measurements 402 and temperature measurements may be obtained in any means as is known in the art. Such known means in the art may include the use of a four electrode system made of a conductive metal, such as platinum. Temperature measurements may be made by a high-grade thermometer in one non-limiting embodiment. The two inputs of the electrical conductivity measurements 402 and temperature measurements 404 are provided to step 406 where, with a water salinity of zero, an oil-in-water fraction is calculated.

After 406, data from 406 is fed to three different models based on FIGS. 1 through 3 above. These three models 409 produce data. This data is used to calculate a water salinity value 414 and an oil-in-water fraction 412. Values for oil-in-water fraction 408 are used to calculate the water salinity value 414. Water salinity 410 is used to calculate the oil-in-water fraction 412.

As will be understood, methods performed in embodiments may be accomplished through action of a computer, programmed to perform tasks. The methods performed may be stored in a non-volatile computer memory. Example embodiments of non-volatile computer memory systems may include, but not be limited to a universal serial bus device, a solid-state memory system, a computer memory system or a compact disk. Such embodiments may be configured as an object of manufacture.

Aspects of the disclosure may also use artificial intelligence systems in order to perform method steps. The artificial intelligence may be configured such that the artificial intelligence may be multi-layer, multi-nodal systems. Training of the artificial intelligence system may be trained, in some embodiments, through use of synthetic data. In other embodiments, actual data or field data may be used. In some aspects of the disclosure, deep learning capabilities may be used. In using artificial intelligence, connections may be established such that automatic actuation of other machines or components is accomplished. Such machines may include hydrocarbon recovery equipment, logging equipment and computing equipment.

Aspects of the disclosure, when performed on a computer, may be enabled to be performed on a computer server, a laptop computer, a mobile computing apparatus or other system. These systems may be enabled to interact or be performed upon a web service, the internet, a computer server, a cloud computing network, or other similar system.

Example embodiments of the claims will be disclosed. The example embodiments disclosed should not be considered limiting. In one example embodiment, a method is disclosed comprising obtaining at least one electrical conductivity measurement for a downhole sample. The method may further comprise obtaining at least one temperature measurement for the downhole sample. The method may further comprise calculating an oil-in-water fraction for a water salinity value of zero. The method may further comprise using the oil-in-water fraction for a water salinity value of zero, running three models to produce a first set of data, a second set of data, and a third set of data, wherein the first set of data is related to a first model, the second set of data is related to a second model, and the third set of data is related to a third model. The method may further comprise calculating a final oil-in-water fraction using the first set of data, the second set of data, and the third set of data. The method may also comprise calculating a final water salinity using the first set of data, the second set of data, and the third set of data.

In another example embodiment, the method may be performed wherein the calculating the final oil-in-water fraction also uses a salinity value.

In another example embodiment, the method may be performed wherein the calculating the final salinity also uses an oil-in-water fraction.

In another example embodiment, the method may be performed wherein the at least one electrical conductivity measurement is performed in a downhole environment.

In another example embodiment, the method may be performed wherein the at least one temperature measurement is performed in a downhole environment.

In another example embodiment, the method may further comprise storing the at least one of the final salinity and the final oil-in-water values in a non-volatile memory.

In another example embodiment, the method may further comprise displaying the at least one of the final salinity and the final oil-in-water values on a monitor.

In another example embodiment, the method may be performed wherein the first model is related to values for a graph of oil-in-water data in relation to conductivity measurements.

In another example embodiment, the method may be performed wherein the second model is related to a graph of normalized conductivity versus water phase salinity.

In another example embodiment, the method may be performed wherein the third model is related to a graph of interpolation and extrapolation results of electrical conductivity plotted by oil fraction.

In another example embodiment, an object of manufacture configured with a non-volatile memory is disclosed wherein the non-volatile memory is configured to store a list of instructions, the list of instructions configured to be read by a computer, the list of instructions comprising, at least in part, a method. The method may comprise obtaining at least one electrical conductivity measurement for a downhole sample. The method may also comprise obtaining at least one temperature measurement for the downhole sample. The method may also comprise calculating an oil-in-water fraction for a water salinity value of zero. The method may also comprise using the oil-in-water fraction for a water salinity value of zero, running three models to produce a first set of data, a second set of data, and a third set of data, wherein the first set of data is related to a first model, the second set of data is related to a second model, and the third set of data is related to a third model. The method may also comprise calculating a final oil-in-water fraction using the first set of data, the second set of data, and the third set of data. The method may also comprise calculating a final water salinity using the first set of data, the second set of data, and the third set of data.

In another example embodiment, a method is disclosed. In this example embodiment, the method may comprise obtaining at least one electrical conductivity measurement for a downhole sample. The method may also comprise obtaining at least one temperature measurement for the downhole sample. The method may also comprise calculating an oil-in-water fraction for a water salinity value of zero. The method may also comprise using the oil-in-water fraction for a water salinity value of zero, running three models to produce a first set of data, a second set of data, and a third set of data, wherein the first set of data is related to a first model, the second set of data is related to a second model, and the third set of data is related to a third model. The method may also comprise calculating a final oil-in-water fraction using the first set of data, the second set of data, and the third set of data, and a water salinity value. The method may also comprise calculating a final water salinity using the first set of data, the second set of data, and the third set of data, and a oil-in-water fraction.

In another example embodiment, the method may be performed wherein the first model is related to values for a graph of oil-in-water data in relation to conductivity measurements.

In another example embodiment, the method may be performed wherein the second model is related to a graph of normalized conductivity versus water phase salinity.

In another example embodiment, the method may be performed wherein the third model is related to a graph of interpolation and extrapolation results of electrical conductivity plotted by oil fraction.

In another example embodiment, the method may further comprise at least one of displaying or saving the final oil-in-water fraction and final salinity.

In another example embodiment, the method may be performed wherein the obtaining at least one electrical conductivity measurement and the obtaining at least one temperature measurement for the downhole sample are performed downhole.

In another example embodiment, the method may be performed wherein the method is accomplished at a field location.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

1. A method, comprising:
obtaining at least one electrical conductivity measurement for a sample;
obtaining at least one temperature measurement for the sample;
calculating an oil-in-water fraction for a water salinity value of zero;
using the oil-in-water fraction for a water salinity value of zero, running three models to produce a first set of data, a second set of data, and a third set of data, wherein the first set of data is related to a first model, the second set of data is related to a second model, and the third set of data is related to a third model;
calculating a final oil-in-water fraction using the first set of data, the second set of data, and the third set of data; and
calculating a final water salinity using the first set of data, the second set of data, and the third set of data.

2. The method according to claim 1, wherein the calculating the final oil-in-water fraction also uses a salinity value.

3. The method according to claim 1, wherein the calculating the final salinity also uses an oil-in-water fraction.

4. The method according to claim 1, wherein the at least one electrical conductivity measurement is performed in a downhole environment.

5. The method according to claim 1, wherein the at least one temperature measurement is performed in a downhole environment.

6. The method according to claim 1, further comprising storing the at least one of the final salinity and the final oil-in-water values in a non-volatile memory.

7. The method according to claim 1, further comprising displaying the at least one of the final salinity and the final oil-in-water values on a monitor.

8. The method according to claim 1, wherein the first model is related to values for a graph of oil-in-water data in relation to conductivity measurements.

9. The method according to claim 1, wherein the second model is related to a graph of normalized conductivity versus water phase salinity.

10. The method according to claim 1, wherein the third model is related to a graph of interpolation and extrapolation results of electrical conductivity plotted by oil fraction.

11. An object of manufacture configured with a non-volatile memory,
the non-volatile memory configured to store a list of instructions, the list of instructions configured to be read by a computer, the list of instructions comprising, at least in part, a method comprising:
obtaining at least one electrical conductivity measurement for a sample;
obtaining at least one temperature measurement for the sample;
calculating an oil-in-water fraction for a water salinity value of zero;
using the oil-in-water fraction for a water salinity value of zero, running three models to produce a first set of data, a second set of data, and a third set of data, wherein the first set of data is related to a first model, the second set of data is related to a second model, and the third set of data is related to a third model;
calculating a final oil-in-water fraction using the first set of data, the second set of data, and the third set of data; and
calculating a final water salinity using the first set of data, the second set of data, and the third set of data.

12. The article of manufacture wherein the article is configured as one of a solid-state device, a universal serial bus device, a compact disk, and a computer memory arrangement.

13. A method, comprising:
obtaining at least one electrical conductivity measurement for a sample;
obtaining at least one temperature measurement for the sample;
calculating an oil-in-water fraction for a water salinity value of zero;
using the oil-in-water fraction for a water salinity value of zero, running three models to produce a first set of data, a second set of data, and a third set of data, wherein the first set of data is related to a first model, the second set of data is related to a second model, and the third set of data is related to a third model;
calculating a final oil-in-water fraction using the first set of data, the second set of data, and the third set of data, and a water salinity value; and
calculating a final water salinity using the first set of data, the second set of data, and the third set of data, and a oil-in-water fraction.

14. The method according to claim 12, wherein the first model is related to values for a graph of oil-in-water data in relation to conductivity measurements.

15. The method according to claim 12, wherein the second model is related to a graph of normalized conductivity versus water phase salinity.

16. The method according to claim 12, wherein the third model is related to a graph of interpolation and extrapolation results of electrical conductivity plotted by oil fraction.

17. The method according to claim 12, further comprising at least one of displaying or saving the final oil-in-water fraction and final salinity.

18. The method according to claim 12, wherein the obtaining at least one electrical conductivity measurement and the obtaining at least one temperature measurement for the sample are performed downhole.

19. The method according to claim 12, wherein the method is accomplished at a field location.
